(19) United States
(12) Patent Application Publication
(10) Pub. No.: US 2013/0331354 A1
(43) Pub. Date: Dec. 12, 2013

(54) METHOD AND COMPOSITION UTILIZING LARCH WOOD EXTRACTS IN ANIMAL FEED

(71) Applicants: Sergey V. Philippov, Moscow (RU); Igor M. Bogorodov, Moscow (RU)

(72) Inventors: Sergey V. Philippov, Moscow (RU); Igor M. Bogorodov, Moscow (RU)

(73) Assignee: FLAVITPURE, INC., Cheyenne, WY (US)

(21) Appl. No.: 13/490,621

(22) Filed: Jun. 7, 2012

(51) Int. Cl. A61K 31/715; A61P 1/00; A61P 21/06

(52) U.S. Cl. 514/54

(57) ABSTRACT

A method for improving animal feed efficiency, efficiency of animal digestion, overall health of animals, and animal feed production efficiency. The method includes administering an effective amount of a feed additive composition having a combination of a dietary fiber and a flavonoid to an animal. The animal feed composition includes a Larch Arabinogalactan in an amount between 0.1% and 30% of the animal feed composition, a Dihydroquercetin (taxifolin) in an amount between 0.1% and 10% of the animal feed composition, and an animal feed carrier or an excipient.

### REFERENCES

[1]. Tatjana Stevanovic, Papa Niokhor Diouf and Martha Estrella Garcia-Perez (2009). Bioactive Polyphenols from Healthy Diets and Forest Biomass. Current Nutrition & Food Science, Vol. 5, No. 4, p. 264-295.
[2] Lee S B, Cha K H, Selenge D, Solongo A, Nho C W (2007). The chemopreventive effect of taxifolin is exerted through ARE-dependent gene regulation. Biol Pharm Bull., 30(6): 1074-1079.
[3] Gupta M B, Bhalla T N, Gupta G P, Mitra C R, Bhargava K P. (1971). Anti-inflammatory activity of taxifolin. Japan J Pharmacol., 21(3):377-82.
[4] Hillis W E (1971) Distribution, properties and formation of some wood extractives. Wood Science and Technology 5: 272-289.
[5] Coté W A, Day A C, Simson B W, Timell T E (1966) Studies on larch arabinogalactan 1. The distribution of arabinogalactan in larch wood. Holzforschung 20: 178-192.
[6] S. Willfor, R. Sjoholm, C. Laine, B. Holmbom. (2002) Structural features of water-soluble arabinogalactans from Norway spruce and Scots pine heartwood. Wood Science and Technology, 36: 101-110.
[7] Lewin M. & Goldstein I. S. (1991): Wood structure and composition. Marcel. Dekker, Inc. International fiber science and technology series: vol. 11. ISBN: 0-8247-8233-x
[8] Giwa S. A. O. & Swan E. P. (1975). Heartwood extractives of a western larch tree (Larixoccidentalis Nutt.). Wood and Fiber vol. 7(3), pp. 216-221.
[9] Terziev, N. (2002b): Properties and Processing of Larch Timber—a Review based on Soviet and Russian Literature.
[10]. Pew, John C., 1947. A flavanone from Douglas-fir heartwood. J. Am. Chem. Soc., 70 (9), pp 3031-3034.
[11]. E. F. Kurth, Harry J. Kiefer, and James K. Hubbard, (1948). Utilization of Douglas-fir Bark. The Timberman, Vol. 49, No. 8, pp. 130-1.
[12]. H. M. Graham, E. F. Kurth, (1949). Constituents of Extractives from Douglas Fir. Ind. Eng. Chem., 41. (2), pp 409-414.
[13]. Migita, Nobuhiko,-Nakano, Junzs, Sakai, Isamu, and Ishi, Shoichi, (1952). Japan Tech. Assoc. Pulp Paper Ind. 6:476-480.
[14]. Kurth, E. F., and Chan, F. L. (1953). “Extraction of Tannin and Dihydroquercetin from Douglas Fir Bark.” J. Amer. Leather Chem. Assoc. 48(1):20-32, Abstr. Bull. Inst. Pap. Chem. 23:469.
[15]. G. M. Barton, J. A. F. Gardner. (1958). Determination of Dihydroquercetin in Douglas Fir and Western Larch Wood. Anal. Chem., 30 (2), pp 279-281.
[16]. G. V. Nair and E von Rudloff. (1959). THE CHEMICAL COMPOSITION OF THE HEARTWOOD EXTRACTIVES OF TAMARACK (LARIX LARICINA (DU ROI) K. K0CH)1. Can. J. Chem., Vol. 37, pp. 1608-1613.
[17]. Tyukavkina, N. A., Lapteva, K. I., Larina V. A.(1967). Extractives of Larixdahurica. Quantitative content of quercetin and dihydroquercetin. Chemistry of Natural Substances. Issue 5, pages 298-301.
[18]. Pietarinen S P, Willfor S M, Vikstrom F A, Holmbom B R. (2006) Aspen knots, a rich source of flavonoids. J Wood Chem Technol., 26: 245-58.
[19]. Conde E, Cadahia E. Garciavallejo M, Tomasbarberan F. (1995) Low molecular-weight polyphenols in wood and bark of Eucalyptus globulus. Wood Fiber Sci., 27: 379-83.
[20]. Antonova, G. F. 1980. Zapasi, sostavi svojstva drevesini listvennitzej. In “Issledovaniya v oblasti drevesiny i drevesnykh materialov”. Institut lesaidrevesiny, Krasnoyarsk, 6-18.
[21]. G. V. Nair and E. von Rodloff. (1959). THE CHEMICAL COMPOSITION OF THE HEARTWOOD EXTRACTIVES OF TAMARACK (LARIX LARICINA (DU ROI) K. K0CH). CANADIAN JOURNAL OF CHEMISTRY. VOL. 37. Issued as N.R.C. No. 5295.
[22]. HENRIK OUTTRUP, KJELD SCHAUMBURG and JORGEN OGAARD MADSEN. (1985). ISOLATION OF DIHYDROMYRICETIN AND DIHYDROQUERCETIN FROM. BARK OF PINUS CONTORTA. Carlsberg Res. Commun. Vol. 50, p. 369-379.
[23] Grant-Thompson., W., The Fibre Story, Gut Reactions, Understanding Symptoms in the Digestive Tract: 45 Plenum Press, N.Y., pp. 59 (1989).
[24] McNeil, N. I. (1988). Nutritional implications of human and mammalian large intestinal function. In World Review of Nutrition and Dietetics, vol. 56, pp. 1-42.
[25] A. J. VINCE, N. I. McNEIL, J. D. WAGER AND O. M. WRONG. The effect of lactulose, pectin, arabinogalactan and cellulose on the production of organic acids and metabolism of ammonia by intestinal bacteria in a faecal incubation system. British Journal of Nutrition (1990), 63, 17-26.
[26] Okubo, T., et al., Biosci. 40 Biotech. Biochem., 56(4):588-591 (1992).
[27] Zhao K, Whiteman M. Spencer J, Halliwell B. DNA damage by nitrite and peroxynitrite: protection by dietary phenols. Methods Enzymol 2001; 335:296-307.
[28] van Lieshout E M, Posner G H, Woodard B T, Peters W H. Effects of the sulforaphane analog compound 30, indole-3-carbinol, D-limonene or relafen on glutathione S-transferases and glutathione peroxidase of the rat digestive tract. Biochim Biophys Acta 1998; 1379:325-36.
[29] Halliwell B. Antioxidant activity and other biological effects of flavonoids. In: Rice-Evans C. ed. Wake up to flavonoids. London: Royal Society of Medicine Press, 2000:13-23.
[30] Halliwell B, Gutteridge J M C. Free radicals in biology and medicine. 3rd ed. Oxford, United Kingdom Clarendon Press, 1999.
[31] Blakeborough M H, Owen R W, Bilton R F. Free radical generating mechanisms in the colon: their role in induction and promotion of colorectal cancer? Free Radic Res Commun 1989; 6:359-67.
[32] Chamulitrat W. Activation of the superoxide-generating NADPH oxidase of intestinal lymphocytes produces highly reactive free radicals from sulfite. Free Radic Biol Med 1999; 27:411-21.
[33] Mittra, B. et al., Molecular Medicine, 6, 2000, 527-541.
[34] Odonmazig P, Ebringerova A, Machova E, Alfoldi J. Structural and molecular properties of the arabinogalactan isolated from Mongolian larchwood (Larixdahurica L.): Carbohydr Res 1994; 252:317-324.
U.S. Pat. No. 6,558,693 B1. Inge Helmer Knap at al. ANIMAL FEED ADDITIVES.
U.S. Pat. No. 6,241,983. Paul, Stephen M. at al. Bacteria- and fiber-containing composition for human gastrointestinal health.
U.S. Pat. No. 6,087,092. Geoffrey N. Richards. COMPOSITIONS AND METHODS FOR ANIMAL HUSBANDRY AND FOR TREATING GASTROINTESTINAL DISORDERS.

### FIELD OF THE INVENTION

The present invention is directed to the use of wood extracts or natural compounds in particular antioxidant Dihydroquercetin (taxifolin), non-starch polysaccharide Arabinogalactan and Arabinogalactan in combination with Dihydroquercetin (taxifolin) for applications in animal feed diets, wherein wood extracts are suggested to use as natural compounds for exposing numerous benefits within animal feed diets for improving feed efficiency, efficiency of digestion and overall health of animals, to improve animal health, welfare, and performance/production efficiency.

### BACKGROUND OF THE INVENTION

It is known that the forest biomass is the most important biomass on Earth, and as wood industry is generating the huge amounts of residues, which are available as an important vegetable resource for further processing and valorization of dietary ingredients through extraction. The extractable flavonoids, obtainable by solvent extraction of the forest biomass, are of special interest as they are readily available from different types of forest and wood transformation residues. One of the most notorious bioactive properties of flavonoids are their antioxidant and anti-inflammatory activities. The most important results on antioxidant capacity of forest trees extracts are presented and compared to those obtained for the extracts from healthy foods rich in bioactive flavonoid molecules [1]. Flavonoid Dihydroquercetin (taxifolin) is one of the most effective natural antioxidant and anti-inflammatory compound [2,3]. The emphasis is also put on residues of conifer wood transformation such as butt logs and bark as these materials represent particularly rich resources for mainly of the dietary fiber Arabinogalactan [4,5,6,7,8]. Higher Arabinogalactan content often goes hand in hand with higher amount of flavonoid substances such as Dihydroquercetin (taxifolin) [9], which had been noted in the literature [10-22] to occur in a larch hardwood, including stems, bark and roots.

Before this invention, it has been known that the extracts of different parts of conifer wood species contain a variety of compounds, such as natural antioxidant Dihydroquercetin (taxifolin), natural non-starch polysaccharide, which is a soluble dietary fiber Arabinogalactan from hardwoods, mainly from plant genus Larix, especially from the Larixcajanderi, Larixczekanowskii, Larixdahurica, Larixgmelinii, Larixkamtschatica, Larixrussica, Larixsibirica, Larixsukaczewii. i.e. Larch Arabinogalactan, which can be defined as a fiber containing significant amounts of natural antioxidants, mainly Dihydroquercetin (taxifolin) associated naturally to the fiber matrix with the following specific characteristics: 1. Dietary fiber content, higher than 70% dry matter basis. 2. One gram of dietary fiber Larch Arabinogalactan should have a capacity to inhibit lipid oxidation equivalent to, at least, 1,000 umol TE/gram basing on ORAC value and normally to 2,000-4,000 umol TE/gram 3. One gram of dietary fiber Larch Arabinogalactan should have a capacity of Cell-based Antioxidant Protection (CAP-e) to protect live cells from oxidative damage to, at least 6 CAP-e units per gram, where the CAP-e value is in Gallic Acid Equivalent (GAE) units. 4. The antioxidant capacity must be an intrinsic property, derived from natural constituents of the material (soluble in digestive fluids) not by added antioxidants or by previous chemical or enzymatic treatments. This invention relates to the use of larch wood extracts such as Dihydroquercetin (taxifolin), Arabinogalactan and Arabinogalactan in combination with Dihydroquercetin (taxifolin) for applications in feeding products for animals, wherein larch wood extracts are suggested to use as dietary ingredients, natural antioxidants, feed additives and feed preservatives.

Traditionally animal feed diets are mainly based on cereals and soybean meal. However, the use of alternative products such as peas, beans, sunflower meal, rapeseed meal, lupines, cereal by-products and sugar beet pulp has received increasing interest in recent years. In some of these products, e.g. sunflower meal, rapeseed meal, lupines, cereal by-products and sugar beet pulp, low digestibility often limits their inclusion in appreciable quantities in animal feed diets. This low digestibility is associated with the composition of the carbohydrate fraction in these products, which mainly consists of non-starch polysaccharides. Non-starch polysaccharides are not degraded in the small intestine by the digestive enzymes of monogastric animals, and hence do not offer their full energy potential to the animal. Hydrolysis of these polysaccharides are known to solve two problems, one of animal welfare and the other relating to an improved economy in production.

Farm animals, like humans, are healthiest when they eat certain foods. Cows, have stomachs that are designed to digest grass. Pigs can digest grass, corn, grains, soy and other plants. Chickens and turkeys can eat plants as well as bugs and worms found on the pasture. When animals are fed conventional (or industrial) feed, which can include animal products, antibiotics, and other unnatural substances such as chewing gum and chicken manure, their health is put in jeopardy. And when an animal is unhealthy, the meat and other products made from it will also be less healthy. Gastrointestinal disorders in animals have been treated with dietary fiber. Dietary fiber is a general term covering a number of substances, including cellulose, hemicellulose such as Larch Arabinogalactan, oligosaccharides, pectins, gums, waxes, and lignin. A more general definition is “endogenous components of plant materials in the diet that are resistant to digestion by human or animal(intestinal) enzymes, i.e., mainly non-starch polysaccharides and lignin. Dietary fiber can be either soluble or insoluble.

Dietary fiber resists hydrolysis by human and animal alimentary enzymes, but can be fermented by colonic microflora. In general, soluble fiber is more readily fermented than insoluble fiber. The main physiological effects of these substances are on gastric emptying and colonic transit time, and can result in improved glucose tolerance and decreased starch digestion. The fermentation of dietary fiber results in increased bacterial biomass, increased fecal mass, lowering of intra colonic pH due to production of short chain fatty acids, and production of various gases as metabolic end products [24]. Larch Arabinogalactan as well might have some value in the treatment of porto-systemic encephalopathy, as it will tend to lower ammonia absorption without the drastic purgation which may attend for instance the use of lactulose [25].

Polyphenols (especially flavonoids, for example, compounds with a phenyl-C3-phenyl structure, wherein the phenyl rings are functionalized with, one or more hydroxyl groups) derived from plants have been reported to significantly decrease the amount of Clostridium perfrigens and other Clostridium spp. (putrefactive bacteria), and significantly increase the amount of Bifidobacterium spp. (acid forming bacteria) in human feces [26]. Flavonoids and other phenolic compounds might exert direct protective effects in the gastro-intestinal (GI) tract, by scavenging reactive oxygen and chlorine species. They could inhibit heme protein-induced peroxidation in the stomach. They are able to inhibit. DNA base deamination by HNO2-derived reactive nitrogen species [27]. Phenols might up-regulate toxin-metabolizing or antioxidant defense enzymes in the GI tract [28]. They might chelateredox-active transition metal ions and decrease their prooxidant potential [29,30]. Dietary iron is usually not completely absorbed, especially in a high fat feed diets. Unabsorbed dietary iron enters the feces, where it could represent a prooxidant challenge to the colon and rectum [31]. Indeed, diets rich in fat and low in fiber may aggravate this prooxidant effect [32]. Phenolic compounds, by chelating iron, may help to alleviate prooxidant actions of colonic iron. It has now been discovered that a class of flavonoids is effective in treating parasitic infection in a manner which promises significant benefit in the treatment of mammals, especially humans, cattle, sheep, goats, horses, pigs, poultry, dogs and cats [33]. In particular these flavonoids have the desirable quality of being orally administrable.

U.S. Pat. No. 6,558,693 B discloses animal feed additives comprising enzymes such as arabinogalactanendo-1,4-beta-galactosidase and/or an arabinogalactanendo-1,3-beta-galactosidase.

U.S. Pat. No. 6,241,983 discloses a composition for promoting gastrointestinal health contains an effective amount of a beneficial human intestinal microorganism and an effective amount of dietary fiber.

U.S. Pat. No. 6,087,092 discloses compositions containing hemicelluloses in combination with polyphenols, methods of preparing the compositions, and methods of treating humans or animals with the composition for increasing growth rate, improving feed efficiency and decreasing scour after weaning in an animal by administering an effective amount of the composition to the animal.

### SUMMARY OF THE INVENTION

It is therefore an object of the present invention to provide an inexpensive feed additives in particular larch wood extracts are suggested to use as natural compounds for exposing numerous benefits within animal feed diets for improving feed efficiency, efficiency of digestion and overall health of animals, to improve animal health, welfare, and performance/production efficiency.

It has now been found that a certain wood extracts designated larch wood extracts are particularly beneficial for incorporation into animal feed, in particular when incorporated together by comprising soluble dietary fiber and flavonoid.

Accordingly, in its first aspect, the present invention provides animal feed additives comprising effective amounts of soluble dietary fiber and flavonoid.

In another aspect the invention provides a method of improving the feed efficiency of an animal diet, which method comprises supplementation of the animal feed additive of the invention to monogastric animals.

In yet another aspect, the invention provides a process for pre-treatment of animal feed, by which process the animal feed is subjected to the action of a larch wood extracts.

Preferred animals to be fed include, but are not limited to cows, pigs, poultry, calves, horses and domestic pets. The composition preferably is administered to animals as a feed additive, at a dosage level of between 0.1 to 30% by weight of feed, preferably between 0.1 and 10% by weight of feed. Preferably, the composition is administered to animals as a powder added to feedstuff formulations or to drinks.

### BRIEF DESCRIPTION OF THE DRAWINGS

The present invention is further illustrated by reference to the accompanying drawing, in which:

FIG. 1 depicts steric structure of dihydroquercetine molecule.

FIG. 2 depicts Larch Arabinogalactan component units.

FIG. 3 shows an example of the composition used as a functional feed as premix for dogs.

FIG. 4 shows an example of the composition used as a functional feed as premix for cats.

### DETAILED DESCRIPTION OF THE INVENTION

Before the present composition and methods of use are disclosed and described, it is to be understood that this invention is not limited to the particular examples, process steps, and materials disclosed herein as such process steps and materials may vary somewhat. It is. also to be understood that the terminology used herein is used for the purpose of describing particular embodiments only and is not intended to be limiting since the scope of the present invention will be limited only by the appended claims and equivalents thereof.

The present invention provides an animal feed additive comprising an effective amount of Larch Arabinogalactan and Dihydroquercetin (taxifolin). In a preferred embodiment, this Larch Arabinogalactan is non-starch soluble polysaccharide defined as dietary fiber, prebiotic and extracted mainly from plant genus Larix, especially from the Larixcajanderi, Larixczekanowskii, Larixdahurica, Larixgmelinii, Larixkamtschatica, Larixrussica, Larixsibirica, Larixsukaczewii. In another preferred embodiment, the feed additive includes Dihydroquercetin (taxifolin), a flavonoid defined as antioxidant, anti-inflammatory, anti-microbial compound and extracted mainly from plant genus Larix as described above. In a third preferred embodiment, the animal feed additive comprises effective amounts of Larch Arabinogalactan combined with Dihydroquercetin (taxifolin) defined as a dietary fiber containing significant amounts of natural antioxidants associated naturally with the fiber matrix, wherein the Larch Arabinogalactan is combined with the Dihydroquercetin (taxifolin) extracted mainly from plant genus Larix as described above.

In the context of this invention, an animal feed additive is a larch wood extract preparation comprising flavonoid Dihydroquercetin (taxifolin) and\or non-starch polysaccharide Arabinogalactan and\or Arabinogalactan in combination with Dihydroquercetin (taxifolin) and suitable carriers and/or excipients, and which larch wood extract preparation is provided in a form that is suitable for being added to animal feed. The animal feed additive of the invention may be prepared in accordance with methods known in the art and may be in the form of a dry or a liquid preparation.

In a specific embodiment the animal feed additive of the invention is a granulated larch wood extract which may readily be mixed with feed components, or more preferably, form a component of a premix. The granulated larch wood extract may be coated or un-coated. The particle size of the larch wood extract granulates preferably is compatible with that of feed and premix components. This provides a safe and convenient means of incorporating larch wood extract into feeds.

In another specific embodiment, the animal feed additive of the invention is a stabilized liquid composition, which may be an aqueous or oil-based slurry. The liquid composition may optionally be added to the animal feed composition after pelleting of this composition.

In another preferred embodiment, the present invention provides an animal feed additive, which additive comprises an effective amount Larch Arabinogalactan combined with Dihydroquercetin (taxifolin), in particular feed dietary fiber enhanced by antioxidant, in particular Larch Arabinogalactan, which can be defined as a fiber containing significant amounts of natural antioxidants. mainly Dihydroquercetin (taxifolin) associated naturally to the fiber matrix with the following specific characteristics: 1. Dietary fiber content, higher than 70% dry matter basis. 2. One gram of dietary fiber Larch Arabinogalactan should have a capacity to inhibit lipid oxidation equivalent to, at least, 1,000 umol TE/gram basing on ORAC value and normally to 2,000-4,000 umol TE/gram 3. One gram of dietary fiber Larch Arabinogalactan should have a capacity of Cell-based Antioxidant Protection (CAP-e) to protect live cells from oxidative damage to, at least 6 CAP-e units per gram, where the CAP-e value is in Gallic Acid Equivalent (GAE) units. 4. The antioxidant capacity must be an intrinsic property, derived from natural constituents of the material (soluble in digestive fluids) not by added antioxidants or by previous chemical or enzymatic treatments.

In its most preferred embodiment, the present invention provides an animal feed additive comprising a Larch Arabinogalactan and a Dihydroquercetin (taxifolin) only. In animal feed, flavonoid such as Dihydroquercetin (taxifolin) is attached to Larch Arabinogalactan, a major constituent of the Arabinogalactan matrix.

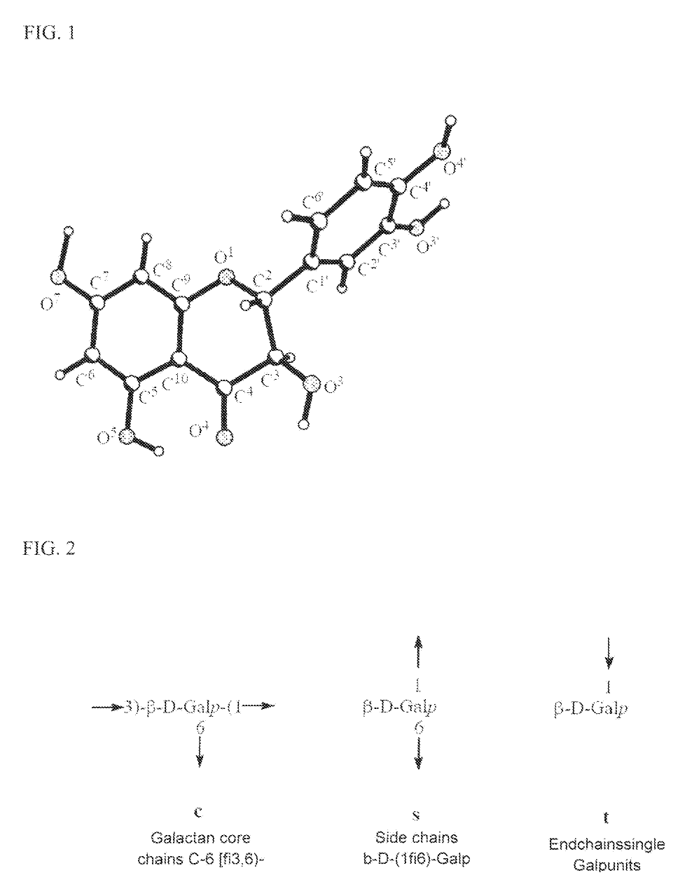

Dihydroquercetin (taxifolin) is the compound having molecule structure is. based on C6-C3-C6 skeleton consisting of two aromatic rings joined by a three Carbon link with the absence of the C2-C3 double bond and have two chiral carbon atoms in position 2 and 3. See FIG. 1. The A ring of the flavonoid structure being acetate derived (3×C2) and the C and B rings originating from cinnamic acid derivatives.(phenylpropanoid pathway). Consequently, the B-ring can be either in the (2S)- or (2R)-configuration. The C-3 atom of dihydroflavonol Dihydroquercetin (taxifolin) bears both a hydrogen atom and a hydroxyl group, and is therefore an additional center of asymmetry. Thus, four stereo isomers are possible for each dihydroflavonol structure, (2R,3R), (2R,3S), (2S,3R), and (2S,3S). All four configurations have been found in naturally occurring dihydroflavonols, but the (2R,3R)-configuration is by far the most common.

Method of extracting the flavonoid compound Dihydroquercetin (taxifolin) suggests extraction is performed in vacuum system of using energy to heat the solvent mixture, a mixture of two or more polar agents in contact with wood particles in order to extract flavonoid Dihydroquercetin (taxifolin) from the wood particles. The extraction conditions including solid/liquid ratio, extraction time, extraction temperature and degree of vacuum, which can be optimized followed by isolation of the flavonoid in question by methods known in the art.

Larch Arabinogalactans are class of long, densely branched low and high-molecular polysaccharides MW: 3,000-120,000. The molecular structures of water-soluble arabinogalactans from different hardwood species have been intensively investigated. Larch Arabinogalactans consist of a main chain of b-D-(1fi3)-galactopyranose units (b-D-(1fi3)-Galp) where most of the main-chain units carry a side chain on C-6 [fi3,6)-Galp-(1fi]. Almost half of these side chains are b-D-(1fi6)-Galp dimers, and about a quarter are single Galp units [FIG. 2]. The rest contain three or more units. Arabinose is present both in the pyranose (Arap) and furanose (Araf) forms, attached to the side chains as arabinobiosyl groups [b-L-Arap-(1fi3)-LAraf-(1fi] or as terminal a-L-Araf e.g. a single L-arabinofuranose unit or 3-O-(β-L-arabinopyranosyl)-α-L-arabinofuranosyl units

Method of extracting polysaccharide substances or Larch Arabinogalactan and Larch Arabinogalactanin combination with Dihydroquercetin (taxifolin) suggests extraction is performed in vacuum system of using energy to heat the solvent mixture, a mixture of two or more polar agents in contact with wood particles in order to extract polysaccharide substances from the wood particles. The extraction conditions including solid/liquid ratio, extraction time, extraction temperature and degree of vacuum, which can be optimized followed by isolation of the polysaccharide substances in question by methods known in the art.

The extraction of Larch Arabinogalactan and Larch Arabinogalactanin combination with flavonoid Dihydroquercetin (taxifolin) from larch wood is very advantageous compared to the utilization of other sources. As a result, about 70-80% of the extract obtained according to above described method may be the polysaccharide agent or agents, suggesting polysaccharide agent consisting with flavonoid agent. Another interesting feature is that a certain substances may be the dominating compound of the derived polysaccharide group of substances. For example, Arabinogalactan may be about 90-98% of the polysaccharides and Dihydroquercetin (taxifolin) may be about 75-85% of the flavonoids derived from larch wood.

The wood extracts contemplated according to the present invention may be derived from any available source from Conifer wood species, especially those from the family of Pinaceae. In a preferred embodiment the wood extracts are derived from a plant genus Larix. Preferably the plant genus Larix is a Larixcajanderi, Larixczekanowskii, Larixdahurica, Larixgmelinii, Larixkamtschatica, Larixrussica, Larixsibirica, Larixsukaczewii. In more preferred embodiments the plant genus Larix is a Larixdahurica, Larixgmelinii, Larixrussica, Larixsibirica, more specifically Larixdahurica, Larixsibirica.

The larch wood extracts as feed additives can be added to animal feed. Animal feeds include but are not limited to cattle feed, poultry feed, swine feed, horse feed, feed for early-weaned calves, and dog and cat food. Examples of feed additives comprising larch wood extracts, i.e., flavonoid Dihydroquercetin (taxifolin) and\or non-starch polysaccharide Arabinogalactan and\or Arabinogalactan in combination with Dihydroquercetin (taxifolin) are any composition/formulation added to food/feed during its manufacture or its preparation for consumption.

Typical dosage ranges are between 0.1 to 30% by weight of the animal feed, preferably between 0.1 and 10% by weight of the animal feed.

Examples of functional feed including pet food compositions are feed intended to supply necessary dietary requirements, as well as treats (e.g., dog biscuits) or other feed supplements. The animal feed comprising the composition according to the invention may be in the form of a dry composition (for example, kibble). semi-moist composition, wet composition, or any mixture thereof. Alternatively or additionally, the animal feed is a supplement, such as a gravy, drinking water, yogurt, powder, suspension, chew, treat (e.g., biscuits) or any other delivery form.

A further aspect of the invention relates to a feed additive or additive composition, such as to be added to one or more edible feed substance (s) or ingredient (s), for example to prepare a feed composition or for supplementation to an existing feed to form a feed composition.

The so-called premixes are examples of animal feed additives of the invention. A premix designates a preferably uniform mixture of one or more micro-ingredients with diluents and/or carrier. Premixes are used to facilitate dispersion of micro-ingredients in a larger mix.

The premix may be in the form of granules or pellets.

In a particular embodiment, Larch Arabinogalactan combined with Dihydroquercetin (taxifolin) in the form in which it is added to the feed, or when being included in a feed additive, is well-defined. The term well-defined means that the Larch Arabinogalactan preparation is at least 50% pure. In other particular embodiments the well-defined Larch Arabinogalactan preparation is at least 60, 65, 70, 75, 80, 85, 88, 90, 92, 94, or at least 95% pure.

Usually fat- and water-soluble vitamins, as well as trace minerals form part of a so-called premix intended for addition to the feed, whereas macro minerals are usually separately added to the feed. Because intake of dietary fiber may adversely affect the absorption of vitamins and minerals in certain mammals it can be desirable to combine the composition with a vitamin and/or mineral supplement or premix.

According to this method, the animal feed additive of the invention is supplemented to the monogastric animal before or simultaneously with the diet. Preferably, the animal feed additive of the invention is supplemented to the monogastric animal simultaneously with the diet. In a more preferred embodiment, the animal feed additive is added to the diet in the form of a granulate or a stabilized liquid.

In another aspect, the invention provides a process for pre-treatment of animal feed, by which process the animal feed is subjected to the action of a larch wood extracts. Preferably the larch wood extract is a Larch Arabinogalactan combined with Dihydroquercetin (taxifolin).

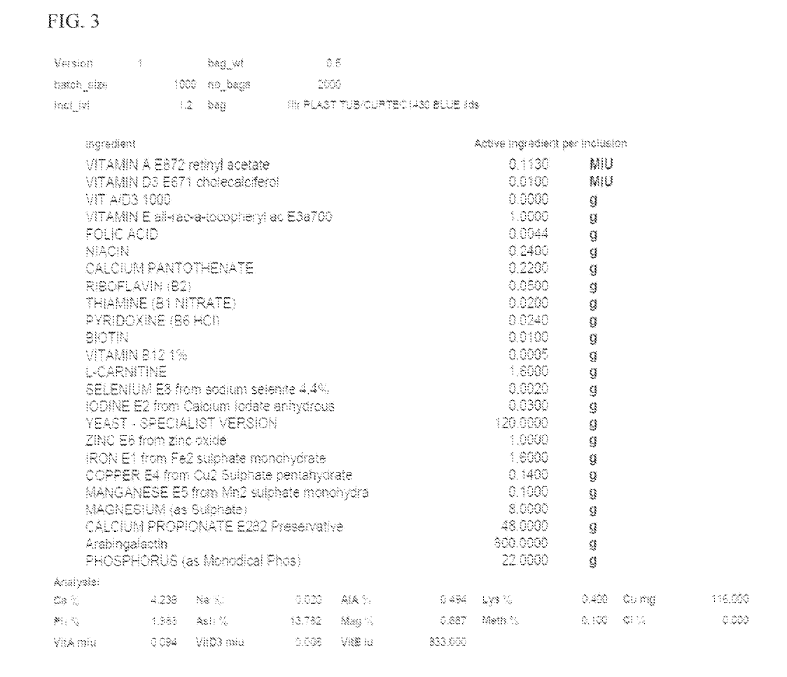

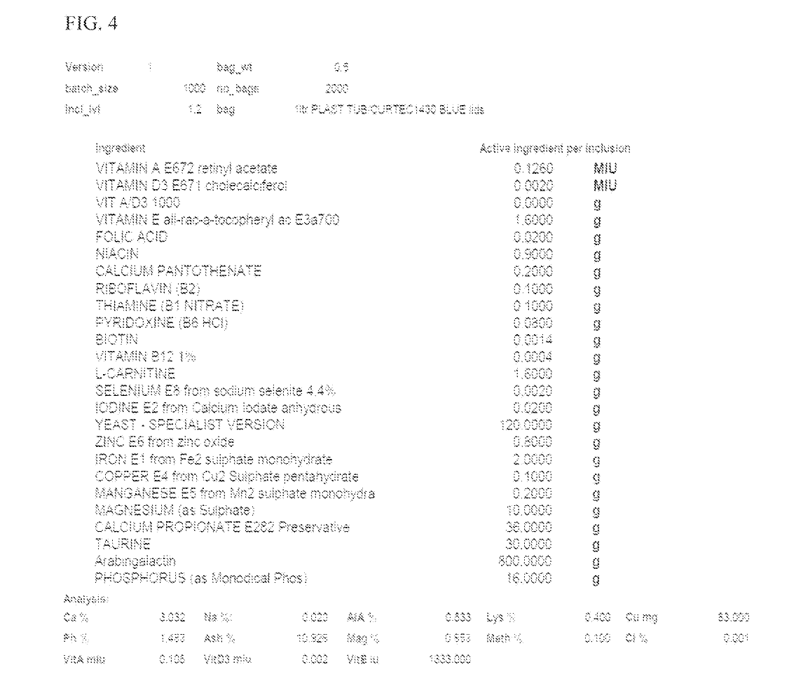

The invention is further illustrated with reference to the two specific embodiments shown in FIGS. 3 and 4, which are not intended to be in any way limiting to the scope of the invention as claimed. Embodiment 1, shown in FIG. 3, shows specific composition used as a functional feed as premix for dogs. Embodiment 2, shown in FIG. 4, shows specific composition used as a functional feed as premix for cats.

This invention has been described with reference to its preferred embodiments. Variations and modifications of the invention will be obvious to those skilled in the art from the foregoing detailed description of the invention. It is intended that all of these variations and modifications be included within the scope of the appended claims.

What is claimed is:

1. An animal feed composition, comprising:
(a) a Larch Arabinogalactan in an amount between 0.1% and 30% of the animal feed composition;
(b) a Dihydroquercetin (taxifolin) in an amount between 0.1% and 10% of the animal feed composition; and
(c) at least one of an animal feed carrier and an excipient.

2. The animal feed composition of claim 1, wherein the Dihydroquercetin (taxifolin) is a flavonoid extracted from at least one of a Larixcajanderi, a Larixczekanowskii, a Larixdahurica, a Larixgmelinii, a Larixkamtschatica, a Larixrussica, a Larixsibirica and a Larixsukaczewii plant.

3. The animal feed composition of claim 1, wherein the Larch Arabinogalactan is a dietary fiber extracted from at least one of a Larixcajanderi, a Larixczekanowskii, a Larixdahurica, a Larixgmelinii, a Larixkamtschatica, a Larixrussica, a Larix sibirica and a Larixsukaczewii plant.

4. The animal feed composition of claim 3, wherein the Larch Arabinogalactan further comprises a second Dihydroquercetin (taxifolin) which is associated naturally with an Arabinogalactan matrix.

5. The animal feed composition of claim 1 wherein the carrier is a feed.

6. A method for improving animal feed efficiency, efficiency of animal digestion, overall health of animals, and animal feed production efficiency, the method comprising the step of:
administering an effective amount of a feed additive composition comprising a combination of a dietary fiber and a flavonoid to an animal.

7. The method of claim 6, wherein the flavonoid is a Dihydroquercetin (taxifolin) extracted from at least one of a Larixcajanderi, a Larixczekanowskii, a Larixdahurica, a Larixgmelinii, a Larixkamtschatica, a Larixrussica, a Larixsibirica, and a Larixsukaczewii plant.

8. The method of claim 6, wherein the dietary fiber is a Larch Arabinogalactan extracted from at least one of a Larixcajanderi, a Larixczekanowskii, a Larixdahurica, a Larixgmelinii, a Larixkamtschatica, a Larixrussica, a Larixsibirica, and a Larixsukaczewii plant.

9. The method of claim 8 wherein the Larch Arabinogalactan further comprises a second Dihydroquercetin (taxifolin), which is associated naturally with an Arabinogalactan matrix.

10. The method of claim 6 wherein the feed additive composition is administered in a dosage of between 0.1% and 30% of the animal feed.

11. The method of claim 6 further comprising a step of adding a carrier to the feed additive composition.

12. The method of claim 11 wherein the carrier is a feed.

13. The method of claim 6 wherein the animal is selected from the group consisting of a cattle, a poultry, a swine, a horse, a calf, a dog and a cat.
